(19) United States
(12) Patent Application Publication
(10) Pub. No.: US 2011/0118314 A1
(43) Pub. Date: May 19, 2011

(54) PIPERIDINE ANALOGS AS GLYCOGEN SYNTHASE ACTIVATORS

(76) Inventor: Weiya Yun, Warren, NJ (US)

(21) Appl. No.: 12/905,327

(22) Filed: Oct. 15, 2010

Related U.S. Application Data
(60) Provisional application No. 61/261,456, filed on Nov. 16, 2009.

(51) Int. Cl. A61K 31/454; C07D 211/06; A61K 31/445; C07D 401/02; C07D 417/12; C07D 207/00; A61K 31/40; A61P 3/00; A61P 3/10

(52) U.S. Cl. 514/326; 546/226; 514/330; 546/208; 546/209; 548/538; 514/423; 546/210

(57) ABSTRACT

Provided herein are compounds of the formula (I):

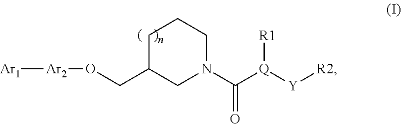

as well as pharmaceutically acceptable salts thereof, wherein the substituents are as those disclosed in the specification. These compounds, and the pharmaceutical compositions containing them, are useful for the treatment of metabolic diseases and disorders such as, for example, type II diabetes mellitus.

### PRIORITY TO RELATED APPLICATION(S)

This application claims the benefit of U.S. Provisional Application No. 61/261,456, filed Nov. 16, 2009, which is hereby incorporated by reference in its entirety.

### FIELD OF THE INVENTION

The invention is directed to compounds, salts and pharmaceutical compositions useful as activators of glycogen synthase for the treatment of metabolic diseases and disorders.

All documents cited or relied upon below are expressly incorporated herein by reference.

### BACKGROUND OF THE INVENTION

Diabetes mellitus is a common and serious disorder, affecting 10 million people in the U.S. [Harris, M. I. Diabetes Care 1998 21 (3S) Supplement, 11C], putting them at increased risk of stroke, heart disease, kidney damage, blindness, and amputation. Diabetes is characterized by decreased insulin secretion and/or an impaired ability of peripheral tissues to respond to insulin, resulting in increased plasma glucose levels. The incidence of diabetes is increasing, and the increase has been associated with increasing obesity and a sedentary life. There are two forms of diabetes: insulin-dependent and non-insulin-dependent, with the great majority of diabetics suffering from the non-insulin-dependent form of the disease, known as type 2 diabetes or non-insulin-dependent diabetes mellitus (NIDDM). Because of the serious consequences, there is an urgent need to control diabetes.

Treatment of NIDDM generally starts with weight loss, a healthy diet and an exercise program. However, these factors are often unable to control the disease, and there are a number of drug treatments available, including insulin, metformin, sulfonylureas, acarbose, and thiazolidinediones. Each of these treatments has disadvantages and there is an ongoing need for new drugs to treat diabetes.

Metformin is an effective agent that reduces fasting plasma glucose levels and enhances the insulin sensitivity of peripheral tissue, mainly through an increase in glycogen synthesis [De Fronzo, R. A. Drugs 1999, 58 Suppl. 1, 29]. Metformin also leads to reductions in the levels of LDL cholesterol and triglycerides [Inzucchi, S. E. JAMA 2002, 287, 360]. However, it loses its effectiveness over a period of years [Turner, R. C. et al. JAMA 1999, 281, 2005].

Thiazolidinediones are activators of the nuclear receptor peroxisome-proliferator activated receptor-gamma. They are effective in reducing blood glucose levels, and their efficacy has been attributed primarily to decreasing insulin resistance in skeletal muscle [Tadayyon, M. and Smith, S. A. Expert Opin. Investig. Drugs 2003, 12, 307]. One disadvantage associated with the use of thiazolidinediones is weight gain.

Sulfonylureas bind to the sulfonylurea receptor on pancreatic beta cells, stimulate insulin secretion, and consequently reduce blood glucose levels. Weight gain is also associated with the use of sulfonylureas [Inzucchi, S. E. JAMA 2002, 287, 360] and, like metformin, they lose efficacy over time [Turner, R. C. et al. JAMA 1999, 281, 2005]. A further problem often encountered in patients treated with sulfonylureas is hypoglycemia [Salas, M. and Caro, J. J. Adv. Drug React. Tox. Rev. 2002, 21, 205-217].

Acarbose is an inhibitor of the enzyme alpha-glucosidase, which breaks down disaccharides and complex carbohydrates in the intestine. It has lower efficacy than metformin or the sulfonylureas, and it causes intestinal discomfort and diarrhea which often lead to the discontinuation of its use [Inzucchi, S. E. JAMA 2002, 287, 360].

Because none of these treatments is effective over the long term without serious side effects, there is a need for new drugs for the treatment of type 2 diabetes.

In skeletal muscle and liver, there are two major pathways of glucose utilization: glycolysis, or oxidative metabolism, where glucose is oxidized to pyruvate; and glycogenesis, or glucose storage, where glucose is stored in the polymeric form glycogen. The key step in the synthesis of glycogen is the addition of the glucose derivative UDP-glucose to the growing glycogen chain, and this step is catalyzed by the enzyme glycogen synthase [Cid, E. et al. J. Biol. Chem. 2000, 275, 33614]. There are two isoforms of glycogen synthase, found in liver [Bai, G. et al. J. Biol. Chem. 1990, 265, 7843] and in other peripheral tissues including muscle [Browner, M. F. et al. Proc. Nat. Acad. Sci. U.S.A. 1989, 86, 1443]. There is clinical and genetic evidence implicating both forms of glycogen synthase in metabolic diseases such as type 2 diabetes and cardiovascular disease. Both basal and insulin-stimulated glycogen synthase activity in muscle cells from diabetic subjects were significantly lower than in cells from lean non-diabetic subjects [Henry, R. R. et al. J. Clin. Invest. 1996, 98, 1231-1236; Nikoulina, S. E. et al. J. Clin. Enocrinol. Metab. 2001, 86, 4307-4314]. Furthermore, several studies have shown that levels of muscle [Eriksson, J. et al. N. Engl. J. Mod. 1989, 331, 337; Schulman, R. G. et al. N. Engl. J. Med. 1990, 332, 223; Thorburn, A. W. et al. J. Clin. Invest. 1991, 87, 489] and liver [Krssak, M. et. al. Diabetes 2004, 53, 3048] glycogen are lower in diabetic patients than in control subjects. In addition, genetic studies have shown associations in several populations between type 2 diabetes and/or cardiovascular disease and mutation/deletion in the GYS1 gene encoding the muscle isoform of glycogen synthase [Orhu-Melander, M. et al. Diabetes 1999, 48, 918; Fredriksson, J. et. al. PLoS ONE 2007, 3, e285; Kolhberg G. et. al. N. Engl. J. Med. 2007, 357, 1507]. Patients lacking GYS2 encoding the liver isoform of glycogen synthase, suffer from fasting ketotic hypoglycemia and postprandial hyperglycemia, hyperlactanemia and hyperlipidemia, supporting the essential role of liver GS in maintaining normal nutrient metabolism. [Weinstein, D. A. et. al. Mol. Genetics and Metabolism, 2006, 87, 284]

Glycogen synthase is subject to complex regulation, involving phosphorylation in at least nine sites [Lawrence, J. C., Jr. and Roach, P. J. Diabetes 1997, 46, 541]. The dephosphorylated form of the enzyme is active. Glycogen synthase is phosphorylated by a number of enzymes of which glycogen synthase kinase 3P (GSK3(3) is the best understood [Tadayyon, M. and Smith, S. A. Expert Opin. Investig. Drugs 2003, 12, 307], and glycogen synthase is dephosphorylated by protein phosphatase type I (PP1) and protein phosphatase type 2A (PP2A). In addition, glycogen synthase is regulated by an endogenous ligand, glucose-6-phosphate which allosterically stimulates the activity of glycogen synthase by causing a change in the conformation of the enzyme that renders it more susceptible to dephosphorylation by the protein phosphatases to the active form of the enzyme [Gomis, R. R. et al. J. Biol. Chem. 2002, 277, 23246].

Several mechanisms have been proposed for the effect of insulin in reducing blood glucose levels, each resulting in an increase in the storage of glucose as glycogen. First, glucose uptake is increased through recruitment of the glucose transporter GLUT4 to the plasma membrane [Holman, G. D. and Kasuga, M. Diabetologia 1997, 40, 991]. Second, there is an increase in the concentration of glucose-6-phosphate, the allosteric activator of glycogen synthase [Villar-Palasi, C. and Guinovart, J. J. FASEB J. 1997, 11, 544]. Third, a kinase cascade beginning with the tyrosine kinase activity of the insulin receptor results in the phosphorylation and inactivation of GSK313, thereby preventing the deactivation of glycogen synthase [Cohen, P. Biochem. Soc. Trans. 1993, 21, 555; Yeaman, S. J. Biochem. Soc. Trans. 2001, 29, 537].

Because a significant decrease in the activity of glycogen synthase has been found in diabetic patients, and because of its key role in glucose utilization, the activation of the enzyme glycogen synthase holds therapeutic promise for the treatment of metabolic diseases such as type 2 diabetes and cardiovascular diseases. The only known allosteric activators of the enzyme are glucose-6-phosphate [Leloir, L. F. et al. Arch. Biochem. Biophys. 1959, 81, 508] and glucosamine-6-phosphate [Virkamaki, A. and Yki-Jarvinen, H. Diabetes 1999, 48, 1101].

The following biaryloxymethylarenecarboxylic acids are reported to be commercially available from Otava, Toronto, Canada, Akos Consulting & Solutions, Steinen, Germany or Princeton BioMolecular Research, Monmouth Junction, N.J.: 4-(biphenyl-4-yloxymethyl)-benzoic acid, 3-(biphenyl-4-yloxymethyl)-benzoic acid, [4-(biphenyl-4-yloxymethyl)-phenyl]-acetic acid, [4-(4′-methyl-biphenyl-4-yloxymethyl)-phenyl]-acetic acid, 4-(4′-methyl-biphenyl-4-yloxymethyl)-benzoic acid, 3-(3-bromo-biphenyl-4-yloxymethyl)-benzoic acid, [4-(3-bromo-biphenyl-4-yloxymethyl)-phenyl]-acetic acid, 2-(4′-methyl-biphenyl-4-yloxymethyl)-benzoic acid, 5-(biphenyl-4-yloxymethyl)-furan-2-carboxylic acid, 5-(4′-methyl-biphenyl-4-yloxymethyl)-furan-2-carboxylic acid, 5-(3-bromo-biphenyl-4-yloxymethyl)-furan-2-carboxylic acid, 4-(biphenyl-4-yloxymethyl)-5-methyl-furan-2-carboxylic acid, 5-methyl-4-(4′-methyl-biphenyl-4-yloxymethyl)-furan-2-carboxylic acid, 4-(3-bromo-biphenyl-4-yloxymethyl)-5-methyl-furan-2-carboxylic acid, 2-(biphenyl-4-yloxymethyl)-4-methyl-thiazole-5-carboxylic acid, [2-(biphenyl-4-yloxymethyl)-thiazol-4-yl]-acetic acid, [2-(4′-methyl-biphenyl-4-yloxymethyl)-thiazol-4-yl]-acetic acid and [5-(biphenyl-4-yloxymethyl)-[1,3,4]oxadiazol-2-yl]-acetic acid.

Some biaryloxymethylarenecarboxylic acids are known in the art. However, none of these known compounds have been associated with either the treatment of diseases mediated by the activation of the glycogen synthase enzyme or to any pharmaceutical composition for the treatment of diseases mediated by the activation of the glycogen synthase enzyme. Andersen, H. S. et al. WO 9740017 discloses the structure and synthetic route to 3-(biphenyl-4-yloxymethyl)-benzoic acid as an intermediate in the synthesis of SH2 inhibitors. Winkelmann, E. et al. DE 2842243 discloses 5-(biphenyl-4-yloxymethyl)-thiophene-2-carboxylic acid as a hypolipemic agent. Mueller, T. et al. DE 4142514 discloses 2-(biphenyl-3-yloxymethyl)-benzoic acid as a fungicide. Ghosh, S. S. et al. WO 2004058679 discloses biaryloxymethylarene acids as ligands of adenine nucleoside translocase. Van Zandt, M. C. WO 2008033455 discloses biphenyl and heteroarylphenyl derivatives as protein phosphatase-1B inhibitors.

Glycogen synthase activators and stimulators of glycogen production have been reported. Chu, C. A et al. US 20040266856 discloses biaryoxymethylarenecarboxylic acids as glycogen synthase activators. Chu, C. A. WO 2005000781 discloses biaryloxymethylarene carboxylic acids as activators of glycogen synthase. Yang, S-P. and Huang, Y. US 20050095219 discloses hyaluronic acid compounds that stimulate glycogen production. Gillespie, P. et al. WO 2005075468 discloses biaryoxymethylarene carboxylic acids as glycogen synthase activators. Gillespie, P. et al. WO 2006058648 discloses biaryoxymethylarene carboxylic acids as glycogen synthase activators. Bucala, R. et al. WO 2007044622 discloses macrophage migration inhibitory factor agonists that stimulate glycogen production.

### SUMMARY OF THE INVENTION

The present invention is directed to compounds of the formula I:

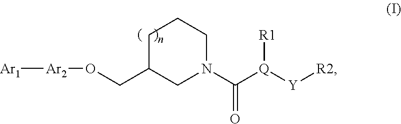

as well as pharmaceutically acceptable salts thereof, pharmaceutical compositions containing them and to methods of treating diseases and disorders. The compounds and compositions disclosed herein are glycogen synthase activators and are useful for the treatment of metabolic diseases and disorders, preferably diabetes mellitus, more preferably type II diabetes mellitus.

### DETAILED DESCRIPTION OF THE INVENTION

In an embodiment of the present invention, provided is a compound of Formula (I):

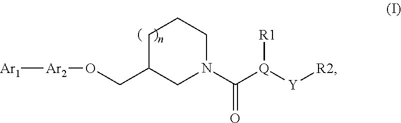

wherein:
Ar₁ is phenyl, mono-, bi- or tri-substituted independently with halogen, lower alkyl or alkoxy;
Ar₂ is phenyl unsubstituted or substituted with halogen;
Q is CH, N, unsubstituted cycloalkyl, unsubstituted heterocycloalkyl or dioxidoisothiazolidine;
Y is CH₂, carbonyl or absent;
R1 is H, lower alkyl, unsubstituted or mono-, bi- or tri-substituted with halogen, —NR3R4, C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1,
   or a pharmaceutically acceptable salt thereof.

Preferably, Ar₁ is phenyl, mono-, bi- or tri-substituted independently with halogen, lower alkyl or alkoxy; Ar₂ is phenyl unsubstituted or substituted with halogen; Q is CH; Y is CH₂, carbonyl or absent; R1 is H, unsubstituted lower alkyl, —NR3R4, —C(O)OC(CH₃)₃, —C(O)CH₃, —C(O)NH₂, —CH₂-phenyl or absent; R2 is H, —NOCH₃, —NOH, —C(O)NH₂, —(CH₂)ₘCOOH, —C(O)NSO₂CH₃ or 1H-tetrazole; R3 is H or lower alkyl; R4 is —C(O)CH₃ or —C(O)OC(CH₃)₃; n is 0 or 1; and m is 0 or 1.

Preferably, Ar₁ is phenyl, mono-, bi- or tri-substituted independently with halogen, lower alkyl or alkoxy; Ar₂ is phenyl unsubstituted or substituted with halogen; Q is N; Y is CH₂, carbonyl or absent; R1 is H, unsubstituted lower alkyl, —NR3R4, —C(O)OC(CH₃)₃, —C(O)CH₃, —C(O)NH₂,

—CH₂-phenyl or absent; R2 is H, —NOCH₃, —NOH, —C(O)NH₂, —(CH₂)ₘCOOH, —C(O)NSO₂CH₃ or 1H-tetrazole; R3 is H or lower alkyl; R4 is —C(O)CH₃ or —C(O)OC(CH₃)₃; n is 0 or 1; and
m is 0 or 1.

Preferably, Ar₁ is phenyl, mono-, bi- or tri-substituted independently with halogen, lower alkyl or alkoxy; Ar₂ is phenyl unsubstituted or substituted with halogen; Q is unsubstituted cycloalkyl;

Y is CH₂, carbonyl or absent; R1 is H, unsubstituted lower alkyl, —NR3R4, —C(O)OC(CH₃)₃, —C(O)CH₃, —C(O)NH₂, —CH₂-phenyl or absent; R2 is H, —NOCH₃, —NOH, —C(O)NH₂, —(CH₂)ₘCOOH, —C(O)NSO₂CH₃ or 1H-tetrazole; R3 is H or lower alkyl; R4 is —C(O)CH₃ or —C(O)OC(CH₃)₃; n is 0 or 1; and m is 0 or 1.

Preferably, Ar₁ is phenyl, mono-, bi- or tri-substituted independently with halogen, lower alkyl or alkoxy; Ar₂ is phenyl unsubstituted or substituted with halogen; Q is unsubstituted heterocycloalkyl; Y is CH₂, carbonyl or absent; R1 is H, unsubstituted lower alkyl, —NR3R4, —C(O)OC(CH₃)₃, —C(O)CH₃, —C(O)NH₂, —CH₂-phenyl or absent; R2 is H, —NOCH₃, —NOH, —C(O)NH₂, —(CH₂)ₘCOOH, —C(O)NSO₂CH₃ or 1H-tetrazole; R3 is H or lower alkyl;

### R4 is —C(O)CH₃ or —C(O)OC(CH₃)₃; n is 0 or 1; and m is 0 or 1.

Preferably, Ar₁ is difluoromethoxy phenyl.

Preferably, Ar₂ is unsubstituted phenyl.

Preferably, Q is CH or N.

Preferably, Q is N.

Preferably, Q is piperidine.

Preferably, Y is CH₂.

Preferably, Y is carbonyl or absent.

Preferably, R1 is H, lower alkyl, tert-butoxycarbonylamino, acetylamino, acetyl-methyl-amino, —C(O)OC(CH₃)₃, —C(O)CH₃, —C(O)NH₂, —CH₂-phenyl or absent.

Preferably, R1 is H.

Preferably, R1 is methyl, ethyl or tert-butyl.

Preferably, R1 is tert-butoxycarbonylamino, acetylamino or acetyl-methyl-amino. Preferably, R1 is —C(O)OC(CH₃)₃, —C(O)CH₃, —C(O)NH₂ or —CH₂-phenyl.

Preferably, R1 is absent.

Preferably, R2 is —NOCH₃, —NOH, —C(O)NH₂, —(CH₂)ₘCOOH, —C(O)NSO₂CH₃ or 1H-tetrazole.

Preferably, R3 is H, methyl or ethyl.

Preferably, R4 is —C(O)CH₃.

Preferably, R4 is —C(O)OC(CH₃)₃.

Preferably, n is 0.

Preferably, n is 1.

Preferably, m is 0.

Preferably, m is 1.

Preferably, the compound according to Formula (I) is:
1-[3-(4′,5′-Difluoro-2′-methoxy-biphenyl-4-yloxymethyl)-piperidine-1-carbonyl]-cyclopropanecarboxylic acid;
3-[3-(4′,5′-Difluoro-2′-methoxy-biphenyl-4-yloxymethyl)-piperidin-1-yl]-3-oxo-propionic acid;
3-[(S)-3-(4′,5′-Difluoro-2′-methoxy-biphenyl-4-yloxymethyl)-piperidin-1-yl]-3-oxo-propionic acid;
3-[(R)-3-(4′,5′-Difluoro-2′-methoxy-biphenyl-4-yloxymethyl)-piperidin-1-yl]-3-oxo-propionic acid;
3-[3-(4′,5′-Difluoro-2′-methoxy-biphenyl-4-yloxymethyl)-piperidin-1-yl]-N-methoxy-3-oxo-propionamide;
3-[3-(4′,5′-Difluoro-2′-methoxy-biphenyl-4-yloxymethyl)-piperidin-1-yl]-N-hydroxy-3-oxo-propionamide;
2-[3-(4′,5′-Difluoro-2′-methoxy-biphenyl-4-yloxymethyl)-piperidine-1-carbonyl]-cyclopentanecarboxylic acid;
(R)(+)-4-[3-(4′,5′-Difluoro-2′-methoxy-biphenyl-4-yloxymethyl)-piperidin-1-yl]-3-methyl-4-oxo-butyric acid;
3-(4′,5′-Difluoro-2′-methoxy-biphenyl-4-yloxymethyl)-piperidine-1-carboxylic acid amide;
(R)-3-tert-Butoxycarbonylamino-4-[3-(4′,5′-difluoro-2′-methoxy-biphenyl-4-yloxymethyl)-piperidin-1-yl]-4-oxo-butyric acid;
(R)-3-Acetylamino-4-[3-(4′,5′-difluoro-2′-methoxy-biphenyl-4-yloxymethyl)-piperidin-1-yl]-4-oxo-butyric acid;
(S)-3-tert-Butoxycarbonylamino-4-[3-(4′,5′-difluoro-2′-methoxy-biphenyl-4-yloxymethyl)-piperidin-1-yl]-4-oxo-butyric acid;
(S)-3-(Acetyl-methyl-amino)-4-[3-(4′,5′-difluoro-2′-methoxy-biphenyl-4-yloxymethyl)-piperidin-1-yl]-4-oxo-butyric acid;
4-[3-(4′,5′-Difluoro-2′-methoxy-biphenyl-4-yloxymethyl)-piperidine-1-carbonyl]-pyrrolidine-1,3-dicarboxylic acid 1-tert-butyl ester;
1-Acetyl-4-[3-(4′,5′-difluoro-2′-methoxy-biphenyl-4-yloxymethyl)-piperidine-1-carbonyl]-pyrrolidine-3-carboxylic acid;
1-Carbamoyl-4-[3-(4′,5′-difluoro-2′-methoxy-biphenyl-4-yloxymethyl)-piperidine-1-carbonyl]-pyrrolidine-3-carboxylic acid;
3-[3-(4′,5′-Difluoro-2′-methoxy-biphenyl-4-yloxymethyl)-piperidin-1-yl]-2-methyl-3-oxo-propionic acid;
2-[3-(4′,5′-Difluoro-2′-methoxy-biphenyl-4-yloxymethyl)-piperidine-1-carbonyl]-butyric acid;
2-[3-(4′,5′-Difluoro-2′-methoxy-biphenyl-4-yloxymethyl)-piperidine-1-carbonyl]-3,3-dimethyl-butyric acid; or
3-[3-(4′,5′-Difluoro-2′-methoxy-biphenyl-4-yloxymethyl)-piperidin-1-yl]-3-oxo-propionamide.

Preferably, the compound according to Formula (I) is:
[3-(4′,5′-Difluoro-2′-methoxy-biphenyl-4-yloxymethyl)-piperidin-1-yl]-(1,1-dioxido-3-isothiazolidin-3-yl)-methanone;
{Benzyl-[3-(4′,5′-difluoro-2′-methoxy-biphenyl-4-yloxymethyl)-piperidine-1-carbonyl]-amino}-acetic acid;
{Benzyl-[(R)-3-(4′,5′-difluoro-2′-methoxy-biphenyl-4-yloxymethyl)-piperidine-1-carbonyl]-amino}-acetic acid;
{Benzyl-[(S)-3-(4′,5′-difluoro-2′-methoxy-biphenyl-4-yloxymethyl)-piperidine-1-carbonyl]-amino}-acetic acid;
{[3-(4′,5′-Difluoro-2′-methoxy-biphenyl-4-yloxymethyl)-piperidine-1-carbonyl]-methyl-amino}-acetic acid;
{[3-(4′,5′-Difluoro-2′-methoxy-biphenyl-4-yloxymethyl)-pyrrolidine-1-carbonyl]-methyl-amino}-acetic acid;
(S)-1-[3-(4′,5′-Difluoro-2′-methoxy-biphenyl-4-yloxymethyl)-piperidine-1-carbonyl]-pyrrolidine-2-carboxylic acid;
3-{Benzyl-[3-(4′,5′-difluoro-2′-methoxy-biphenyl-4-yloxymethyl)-piperidine-1-carbonyl]-amino}-propionic acid;
3-{Benzyl-[3-(4′,5′-difluoro-2′-methoxy-biphenyl-4-yloxymethyl)-pyrrolidine-1-carbonyl]-amino}-propionic acid;
3-(4′,5′-Difluoro-2′-methoxy-biphenyl-4-yloxymethyl)-piperidine-1-carboxylic acid methyl-(1H-tetrazol-5-ylmethyl)-amide;
3-(4′,5′-Difluoro-2′-methoxy-biphenyl-4-yloxymethyl)-piperidine-1-carboxylic acid (2-methanesulfonylamino-2-oxo-ethyl)-methyl-amide;
(S)-3-(4′,5′-Difluoro-2′-methoxy-biphenyl-4-yloxymethyl)-piperidine-1-carboxylic acid (2-methanesulfonylamino-2-oxo-ethyl)-methyl-amide;
(R)-3-(4′,5′-Difluoro-2′-methoxy-biphenyl-4-yloxymethyl)-piperidine-1-carboxylic acid (2-methanesulfonylamino-2-oxo-ethyl)-methyl-amide;
3-(4′,5′-Difluoro-2′-methoxy-biphenyl-4-yloxymethyl)-piperidine-1-carboxylic acid benzyl-(2-methanesulfonylamino-2-oxo-ethyl)-amide;
3-(4′,5′-Difluoro-2′-methoxy-biphenyl-4-yloxymethyl)-pyrrolidine-1-carboxylic acid benzyl-(2-methanesulfonylamino-2-oxo-ethyl)-amide; or
3-(4′,5′-Difluoro-2′-methoxy-biphenyl-4-yloxymethyl)-pyrrolidine-1-carboxylic acid (2-methanesulfonylamino-2-oxo-ethyl)-methyl-amide.

In another preferred embodiment, provided is a pharmaceutical composition, comprising a therapeutically effective amount of a compound according to formula (I) and a pharmaceutically acceptable carrier and/or adjuvant.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, bornyl, adamantyl, indenyl and the like. In a preferred embodiment, the “cycloalkyl” moieties can optionally be substituted with one, two, three or four substituents with the understanding that said substituents are not, in turn, substituted further unless indicated otherwise in the Examples or claims below. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, pyranyl, morpholinyl, thiomorpholinyl, piperazinyl, piperidinyl, pyrrolidinyl, tetrahydropyranyl, tetrahydrofuranyl, 1,3-dioxanyl, dioxidoisothiazolidine and the like. The heterocycloalkyl groups may be unsubstituted or substituted and attachment may be through their carbon frame or through their heteroatom(s) where appropriate, with the understanding that said substituents are not, in turn, substituted further unless indicated otherwise in the Examples or claims below.

The term “lower alkyl”, alone or in combination with other groups, refers to a branched or straight-chain alkyl radical of one to nine carbon atoms, preferably one to six carbon atoms, most preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl and napthyl.

The alkyl, lower alkyl and aryl groups may be substituted or unsubstituted. When substituted, there will generally be, for example, 1 to 4 substituents present, with the understanding that said substituents are not, in turn, substituted further unless indicated otherwise in the Examples or claims below.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. The heteroaryl group may be substituted independently with one, two, or three substituents, with the understanding that said substituents are not, in turn, substituted further unless indicated otherwise in the Examples or claims below. An example of a heteroaryl is 1H-tetrazole.

As used herein, the term “alkoxy” means alkyl-O—; and “alkoyl” means alkyl-CO—. Alkoxy substituent groups or alkoxy-containing substituent groups may be substituted by, for example, one or more alkyl groups with the understanding that said substituents are not, in turn, substituted further unless indicated otherwise in the Examples or claims below.

As used herein, the term “halogen” means a fluorine, chlorine, bromine or iodine radical, preferably a fluorine, chlorine or bromine radical, and more preferably a fluorine or chlorine radical.

Compounds of formula (I) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula (I) in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Chemicals may be purchased from companies such as for example Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

Definitions as used herein include:

GS is glycogen synthase,
THF is tetrahydrofuran,

### DMF is N,N-dimethylformamide,

### DMA is N,N-dimethylacetamide,

DMSO is dimethylsulfoxide,
DCM is dichloromethane,
DME is dimethoxyethane,
MeOH is methanol,
EtOH is ethanol,
NaOH is sodium hydroxide,
TFA is 1,1,1-trifluoroacetic acid,
HOBT is 1-hydroxybenzotriazole,
HOAT is 1-hydroxy-7azabenzotriazole,
EDCI is 1-[3-(dimethylamino)propyl]-3-ethylcarbodiimide hydrochloride,
DIPEA is diisopropylethylamine,
Boc is tert-butyloxycarbonyl,
DBU is 1,8-diazabicyclo[5,4,0]undec-7-ene,
CDI is 1,1′-carbonyldiimidazole,
Brine is saturated aqueous sodium chloride solution,
TLC is thin layer chromatography,
SFC is supercritical fluid chromatography,
RP HPLC is reversed phase high performance liquid chromatography,
HR-MS is high resolution mass spectrometry,
LC-MS is liquid chromatographic mass spectrometry,
RT is room or ambient temperature.

Compounds of the present invention can be prepared beginning with commercially available starting materials and utilizing general synthetic techniques and procedures known to those skilled in the art. Outlined below are reaction schemes suitable for preparing such compounds. Further exemplification can be found in the specific Examples detailed below.

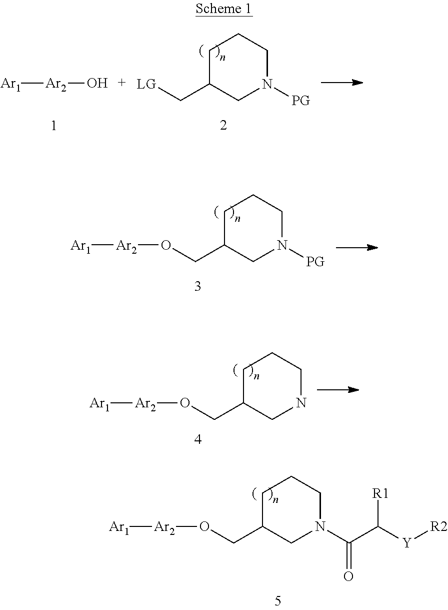

As shown in Scheme 1, above, compounds of the invention can be prepared by nucleophilic displacement of a leaving group LG from a compound of formula 2 by a hydroxybiaryl of formula 1 (compounds of formula 1 are commercially available or can be synthesized according to procedures described in US20040266856) to form a compound of formula 3 in which PG represents a protective group commonly used for the protection of an amino group. The protective group is then cleaved to give the compound of formula 4. Ar₁ is an aryl group, such as phenyl, which can be mono-, bi- or tri-substituted independently with a halogen, lower alkyl or alkoxy group. Ar₂ is an aryl group, such as phenyl, which can be unsubstituted or substituted with halogen. The conversion of compound 4 to compound of formula 5 can be carried out using a variety of procedures that are well known in the field of organic synthesis, and especially well known in the field of peptide synthesis. The compound of formula 4 is reacted with a carboxylic acid, dicarboxylic acid or a suitably mono-protected dicarboxylic acid to give the compound of formula 5 where R2 represents a carboxylic acid or any carboxylic acid mimetics. Examples of such carboxylic acid mimetics are amides, acyl sulfonamides, alkoxyl amides or tetrazole. In the case of R2 is a protected carboxylic acid, subsequent removal of the protection group gives the compound of formula 5 where R2 represents a carboxylic acid.

Many protective groups PG are known to those of skill in the art of organic synthesis. For example, several suitable protective groups are enumerated in “Protective Groups in Organic Synthesis” [Greene, T. W. and Wuts, P. G. M., 2 nd Edition, John Wiley & Sons, N.Y. 1991]. Preferred protective groups are those compatible with the reaction conditions used to prepare compounds of the invention. Examples of such protective groups are carbamates (e.g. t-Butyl carbamate).

The nucleophilic displacement of the leaving group LG in compound 2 can be effected by any conventional means. For example, in the case where LG represents the leaving group chlorine, bromine, or iodine, the reaction can conveniently be carried out by treating compound 2 with compound 1 in the presence of a base such as an alkali metal hydride (for example, sodium hydride) or an alkali metal carbonate (for example, potassium carbonate) in an inert solvent (e.g., N,N-dimethylformamide) at a temperature between about room temperature and about 100° C.

The conversion of compound 3 to compound 4 by deprotection of the carbamate protective group is carried out using reaction conditions that are well known in the field of organic synthesis, and many of which are outlined in “Protective Groups in Organic Synthesis” [Greene, T. W. and Wuts, P. G. M., 2 nd Edition, John Wiley & Sons, N.Y. 1991]. For example, in the case where PG is t-Butyl carbamate, the reaction can be conveniently effected by treating the compound with a strong inorganic acid, for example a hydrohalic acid such as hydrogen chloride or hydrogen bromide, or a strong organic acid, for example a halogenated alkane carboxylic acid such as trifluoroacetic acid and the like, preferably HCl, in a suitable solvent, such as dioxane. The reaction can be carried out at a temperature between about 0° C. and about room temperature, preferably at about room temperature.

A compound of formula 5 where R2 represents a carboxylic acid, a carboxylic acid mimetics or a protected carboxylic acid can be prepared by treating a compound of formula 4 with a carboxylic acid, a dicarboxylic acid or a mono-protected dicarboxylic acid in the presence of a coupling agent, many examples of which are well known per se in peptide chemistry, and in the optional presence of a substance that increases the rate of the reaction, such as 1-hydroxybenzotriazole or 1-hydroxy-7-azabenzo-triazole; or by reaction of the compound of the formula 4 with a reactive derivative of the mono-protected dicarboxylic acid such as the corresponding acid halide (for example, the acid chloride), acid anhydride, activated ester etc. The reaction is conveniently carried out in the presence of a carbodiimide reagent such as n-(3-dimethylaminopropyl)-n′-ethylcarbodiimide hydrochloride in an inert solvent such as methylene chloride, N,N-dimethylformamide or N-methylpyrrolidinone at a temperature between about 0° C. and about room temperature, preferably at about room temperature. The removal of the protective group from the compound of formula 5 in which R2 represents a protected carboxylic acid group can be effected using one of several choices of reactions conditions, the selection of which will depend on the nature of the protective group, and the other functionality present in the compound of formula 5. Many suitable reaction conditions are outlined in “Protective Groups in Organic Synthesis” [T. W. Greene and P. G: M. Wuts, 2nd Edition, John Wiley & Sons, N.Y. 1991]. For example, in the case where the protective group is methyl or ethyl, the reaction can be conveniently effected by treating the compound with one equivalent of an alkali metal hydroxide, such as potassium hydroxide, sodium hydroxide, or lithium hydroxide, preferably lithium hydroxide, in a suitable solvent, such as a mixture of tetrahydrofuran, methanol, and water. The reaction can be carried out at a temperature between about 0° C. and about room temperature, preferably at about room temperature.

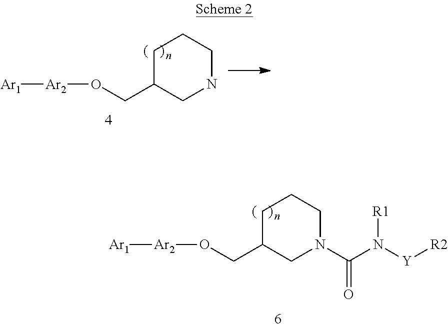

As shown in Scheme 2, above, compound of formula 4 can be converted to compound 6 upon treatment with a suitable isocyanate. Alternatively compound 4 can be treated with phosgene and the like, followed by a reaction with an amine to form a urea compound 6.

Preparative supercritical fluid chromatography (SFC) was performed on Berger Multi Gram II Supercritical Fluid Chromatography system (Model SD-1) from Mettler-Toledo AutoChem Berger Instruments, Newark, Del., USA. The system consisted of an automatic liquid injection system with a DAICEL AD chiral column, 5 mL loop used to make injections and a thermal control module (TCM) used to control column temperature. Chromatographic conditions: SFC separations were performed at a temperature of 30° C., a flow rate of 70 mL/min, and CO₂ pressure of 100 bar. Knauer variable wavelength UV detector (supplied by Mettler-Toledo) with high pressure flow cell was used for SFC detection. Detection in SFC was performed by measurement of UV absorbance at 220 nm.

The invention will now be further described in the Examples below, which are intended as an illustration only and do not limit the scope of the invention.

### EXAMPLES

### Part I: Preparation of Preferred Intermediates 4′,5′-Difluoro-2′-methoxy-biphenyl-4-ol

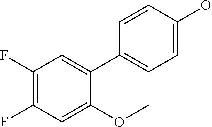

4,5-Difluoro-2-methoxyphenyl-boronic acid (8.8 g, 46.82 mmol) and 4-iodophenol (6.86 g, 31.21 mmol) were suspended in 165 ml of DMF. H₂O (40 mL) was added and the mixture was degassed with argon. Finely ground potassium carbonate (13 g, 93.63 mmol) and tetrakis(triphenylphosphine) palladium(0) (1.5 g, 1.29 mmol) were added. The reaction was stirred at 80-85° C. for 1 hr under argon and cooled. The mixture was diluted with ethyl acetate and water. The organic layer was washed with brine, dried and solvents were evaporated. The crude product was purified by flash chromatography, eluting with 0-8% ethyl acetate in hexanes to yield 4′,5′-difluoro-2′-methoxy-biphenyl-4-ol (6.58 g, 89.3%). LR-MS (ES) calculated for C13H10F2O2, 236.22; found m/z 235 (M−H).

### 3-(4′,5′-Difluoro-2′-methoxy-biphenyl-4-yloxymethyl)-piperidine

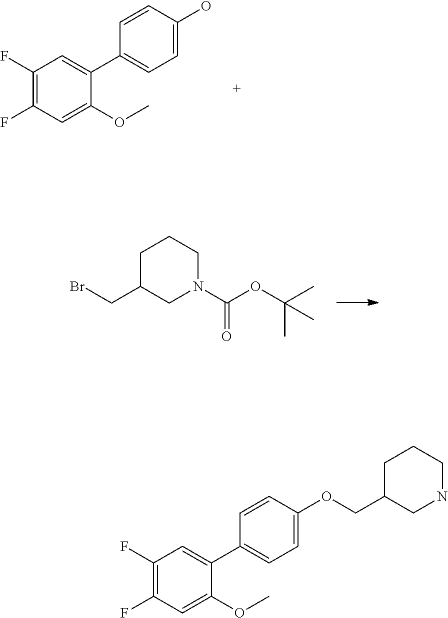

Potassium carbonate (10.2 g, 74 mmol, 6 eq) was added to a solution of 4′,5′-difluoro-2′-methoxy-biphenyl-4-ol (5.72 g, 24.2 mmol) and 3-bromomethyl-piperidine-1-carboxylic acid tert-butyl ester (8.08 g, 29 mmol, 1.2 eq, commercially available) in 120 mL of DMF. The mixture was stirred at 60° C. overnight. The reaction was diluted with water, and extracted with ethyl acetate twice. The organic solution was washed with water and brine, dried over sodium sulfate, and concentrated. The oily residue was then purified by flash chromatography, eluted with 0-40% ethyl acetate in hexanes to afford 8.23 g desired product 3-(4′,5′-difluoro-2′-methoxy-biphenyl-4-yloxymethyl)-piperidine-1-carboxylic acid tert-butyl ester as viscous colorless oil.

3-(4′,5′-difluoro-2′-methoxy-biphenyl-4-yloxymethyl)-piperidine-1-carboxylic acid tert-butyl ester (9.71 g, 22.4 mmol) was treated with 50 mL of 4 M HCl in dioxane in a 250 mL of round bottom flask, and stirred at room temperature for 1 h. The solvent was removed under reduced pressure. The residue was mixed with saturated NaHCO₃ aqueous solution, and extracted with ethyl acetate twice. The organic solution was dried under sodium sulfate, concentrated, and then dried under vacuum. 3-(4′,5′-difluoro-2′-methoxy-biphenyl-4-yloxymethyl)-piperidine was obtained as light yellow viscous oil (6.83 g, 84.7% yield, two steps from the phenol). Mass spectrum [M+H]⁺: 334.

### 3-(4′,5′-Difluoro-2′-methoxy-biphenyl-4-yloxymethyl)-pyrrolidine

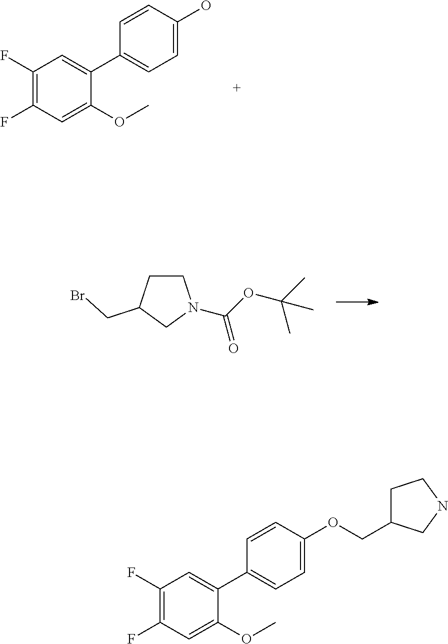

With a method similar as above, 3-(4′,5′-difluoro-2′-methoxy-biphenyl-4-yloxymethyl)-pyrrolidine was prepared from 4′,5′-difluoro-2′-methoxy-biphenyl-4-ol and 3-bromomethyl-pyrrolidine-1-carboxylic acid tert-butyl ester (commercially available). Mass spectrum [M+H]⁺: 320.

### Part II: Preparation of Preferred Embodiments of the Invention

### Example 1

### 1-[3-(4′,5′-Difluoro-2′-methoxy-biphenyl-4-yloxymethyl)-piperidine-1-carbonyl]-cyclopropanecarboxylic acid

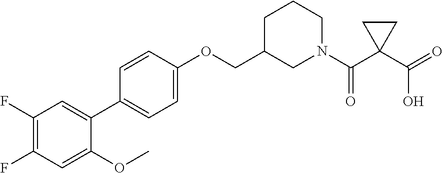

A mixture of 3-(4′,5′-difluoro-2′-methoxy-biphenyl-4-yloxymethyl)-piperidine (100 mg, 0.3 mmol), cyclopropane-1,1-dicarboxylic acid methyl ester (43 mg, 0.3 mmol), 1-hydroxy-7azabenzotriazole (HOAT) (82 mg, 0.6 mmol) and 1-ethyl-2-(3-dimethylaminopropylcarbodiimide hydrochloride (115 mg, 0.6 mmol) in anhydrous dichloromethane (6 mL) was stirred at room temperature overnight. The reaction mixture was mixed with water, and the organic layer was separated. The organic solution was concentrated and purified by flash chromatography eluted with 0-50% ethyl acetate in hexane. The product obtained was treated with excess lithium hydroxide monohydrate (100 mg), and stirred in a mixed solvents of tetrahydrofuan: methanol:water (3:1:1) at room temperature overnight. The reaction mixture was concentrated and mixed with water, acidified with 1 N HCl aqueous solution to pH 1 to 2. The aqueous solution was extracted with ethyl acetate twice. The organic solution was concentrated and dried. The residue was dissolved in 3:1 CH3CN and water, and lyophilized to afford 1-[3-(4′,5′-difluoro-2′-methoxy-biphenyl-4-yloxymethyl)-piperidine-1-carbonyl]-cyclopropane-carboxylic acid as white powder (64 mg, 48% yield). Mass spectrum [M+H]⁺: 446.

### Example 2

### 3-[3-(4′,5′-Difluoro-2′-methoxy-biphenyl-4-yloxymethyl)-piperidin-1-yl]-3-oxo-propionic acid

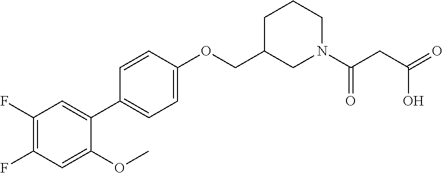

With a method similar as above, 3-[3-(4′,5′-difluoro-2′-methoxy-biphenyl-4-yloxymethyl)-piperidin-1-yl]-3-oxo-propionic acid was prepared from 3-(4′,5′-difluoro-2′-methoxy-biphenyl-4-yloxymethyl)-piperidine and malonic acid monoethyl ester. Mass spectrum [M+H]⁺: 420.

### Example 3

### 3-[(S)-3-(4′,5′-Difluoro-2′-methoxy-biphenyl-4-yloxymethyl)-piperidin-1-yl]-3-oxo-propionic acid (or enantiomer)

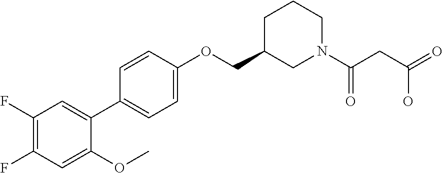

The racemic mixture 3-[3-(4′,5′-difluoro-2′-methoxy-biphenyl-4-yloxymethyl)-piperidin-1-yl]-3-oxo-propionic acid obtained above was separated by chiral SFC to afford 3-[(S)-3-(4′,5′-difluoro-2′-methoxy-biphenyl-4-yloxymethyl)-piperidin-1-yl]-3-oxo-propionic acid (or enantiomer). [α]_{d}²⁵=+29.8 in DMSO, 5 mg/mL.

### Example 4

### 3-[(R)-3-(4′,5′-Difluoro-2′-methoxy-biphenyl-4-yloxymethyl)-piperidin-1-yl]-3-oxo-propionic acid (or enantiomer)

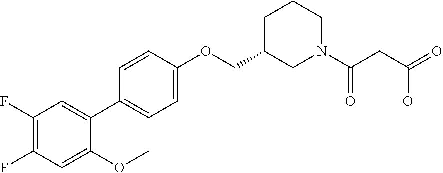

The racemic mixture 3-[3-(4′,5′-difluoro-2′-methoxy-biphenyl-4-yloxymethyl)-piperidin-1-yl]-3-oxo-propionic acid obtained above was separated by chiral SFC to afford 3-[(R)-3-(4′,5′-difluoro-2′-methoxy-biphenyl-4-yloxymethyl)-piperidin-1-yl]-3-oxo-propionic acid (or enantiomer). [α]_{d}²⁵=−19.0 in DMSO, 5 mg/mL.

### Example 5

### 3-[3-(4′,5′-Difluoro-2′-methoxy-biphenyl-4-yloxymethyl)-piperidin-1-yl]-N-methoxy-3-oxo-propionamide

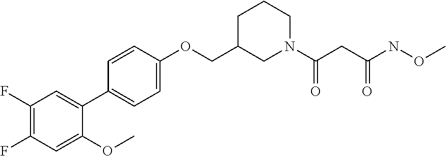

A mixture of 3-[3-(4′,5′-difluoro-2′-methoxy-biphenyl-4-yloxymethyl)-piperidin-1-yl]-3-oxo-propionic acid (50 mg, 0.12 mmol), 1-hydroxy-7azabenzotriazole (HOAT) (33 mg, 0.24 mmol) and 1-ethyl-2-(3-dimethylaminopropylcarbodiimide hydrochloride (46 mg, 0.24 mmol) in anhydrous dichloromethane (2 mL) was stirred at room temperature for 15 min. To this mixture was added a solution of o-methylhydroxylamine hydrochloride (20 mg, 0.24 mmol) and n,n-diisopropylethylamine (0.1 mL) in 1 mL of dichloromethane. The reaction was stirred at room temperature overnight. The reaction mixture was concentrated and purified by flash chromatography eluted with 0-30% methanol in dichloromethane. The product obtained was dissolved in 3:1 CH₃CN and water, and lyophilized to afford 3-[3-(4′,5′-difluoro-2′-methoxy-biphenyl-4-yloxymethyl)-piperidin-1-yl]-N-methoxy-3-oxo-propionamide as white powder (52 mg, 99% yield). Mass spectrum [M+H]⁺: 449.

### Example 6

### 3-[3-(4′,5′-Difluoro-2′-methoxy-biphenyl-4-yloxymethyl)-piperidin-1-yl]-N-hydroxy-3-oxo-propionamide

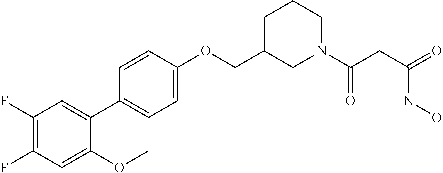

With a method similar as above, 3-[3-(4′,5′-difluoro-2′-methoxy-biphenyl-4-yloxymethyl)-piperidin-1-yl]-N-hydroxy-3-oxo-propionamide was prepared from 3-[3-(4′,5′-difluoro-2′-methoxy-biphenyl-4-yloxymethyl)-piperidin-1-yl]-3-oxo-propionic acid and hydroxylamine. Mass spectrum [M+H]⁺: 435.

### Example 7

### 2-[3-(4′,5′-Difluoro-2′-methoxy-biphenyl-4-yloxymethyl)-piperidine-1-carbonyl]-cyclopentanecarboxylic acid

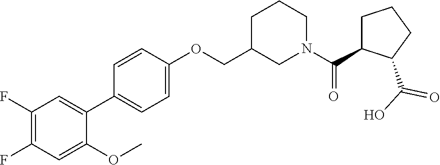

With a method similar as above, 2-[3-(4′,5′-difluoro-2′-methoxy-biphenyl-4-yloxymethyl)-piperidine-1-carbonyl]-cyclopentanecarboxylic acid was prepared from 3-(4′,5′-difluoro-2′-methoxy-biphenyl-4-yloxymethyl)-piperidine and trans-cyclopentane-1,2-dicarboxylic acid monomethyl ester (from Rieke Metals, Inc.). Mass spectrum [M+H]⁺: 474.

### Example 8

### (R)(+)-4-[3-(4′,5′-Difluoro-2′-methoxy-biphenyl-4-yloxymethyl)-piperidin-1-yl]-3-methyl-4-oxo-butyric acid

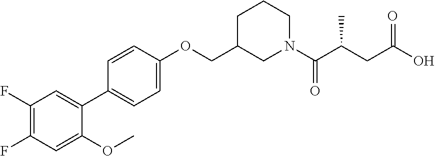

With a method similar as above, (R)(+)-4-[3-(4′,5′-difluoro-2′-methoxy-biphenyl-4-yloxymethyl)-piperidin-1-yl]-3-methyl-4-oxo-butyric acid was prepared from 3-(4′,5′-difluoro-2′-methoxy-biphenyl-4-yloxymethyl)-piperidine and (R)-2-methyl-succinic acid 4-methyl ester. Mass spectrum [M+H]⁺: 448.

### Example 9

### 3-(4′,5′-Difluoro-2′-methoxy-biphenyl-4-yloxymethyl)-piperidine-1-carboxylic acid amide

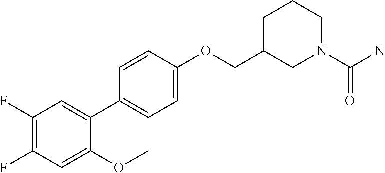

3-(4′,5′-Difluoro-2′-methoxy-biphenyl-4-yloxymethyl)-piperidine (166 mg, 0.5 mmol) was dissolved in anhydrous dichloromethane (5 mL) and treated with trimethylsilyl isocyanate (287 mg, 2.5 mmol, 5 eq). The reaction was stirred at room temperature for overnight. The solvent was removed, and the residue was purified by flash chromatography eluted with 0-30% methanol in dichloromethane. The product obtained was purified again on a thin layer chromatography eluted with 5% methanol in dichloromethane. After lyophilization, 3-(4′,5′-difluoro-2′-methoxy-biphenyl-4-yloxymethyl)-piperidine-1-carboxylic acid amide was obtained as white powder (24 mg, 13% yield). Mass spectrum [M+H]⁺: 377.

### Example 10

### (R)-3-tert-Butoxycarbonylamino-4-[3-(4′,5′-difluoro-2′-methoxy-biphenyl-4-yloxymethyl)-piperidin-1-yl]-4-oxo-butyric acid

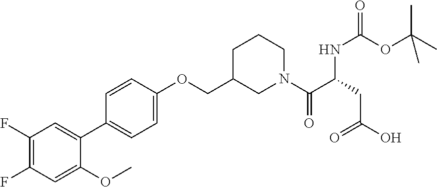

With a method similar as above, (R)-3-tert-butoxycarbonylamino-4-[3-(4′,5′-difluoro-2′-methoxy-biphenyl-4-yloxymethyl)-piperidin-1-yl]-4-oxo-butyric acid was prepared from 3-(4′,5′-difluoro-2′-methoxy-biphenyl-4-yloxymethyl)-piperidine and (R)-2-tert-butoxycarbonylamino-succinic acid 4-benzyl ester. Mass spectrum [M+H]⁺: 549.

### Example 11

### (R)-3-Acetylamino-4-[3-(4′,5′-difluoro-2′-methoxy-biphenyl-4-yloxymethyl)-piperidin-1-yl]-4-oxo-butyric acid

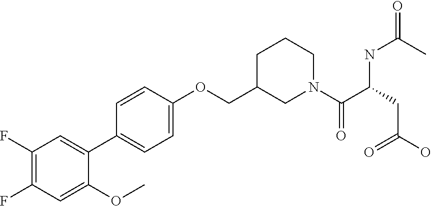

(R)-3-tert-Butoxycarbonylamino-4-[3-(4′,5′-difluoro-2′-methoxy-biphenyl-4-yloxymethyl)-piperidin-1-yl]-4-oxo-butyric acid benzyl ester (100 mg, 0.16 mmol) was treated with 4 mL of 4 N HCl in dioxane, and stirred at room temperature for 1 h. The solvent was removed under reduced pressure. The residue was mixed with saturated NaHCO₃ aqueous solution, and extracted with ethyl acetate twice. The organic solution was concentrated, and then dried under vacuum. The product obtained was then dissolved in 3 mL of dichloromethane and treated with 0.08 mL of triethylamine and 0.08 mL of acetic anhydride. The reaction was stirred at room temperature for 0.5 h and then concentrated. The crude reaction mixture was purified on preparative HPLC. (R)-3-acetylamino-4-[3-(4′,5′-difluoro-2′-methoxy-biphenyl-4-yloxymethyl)-piperidin-1-yl]-4-oxo-butyric acid benzyl ester was obtained as a white solid. It was treated with excess lithium hydroxide monohydrate (20 mg), and stirred in a mixed solvents of tetrahydrofuan: methanol:water (3:1:1) (2 mL) at room temperature for 1 hour. The reaction mixture was concentrated and mixed with water, acidified with 1 N HCl aqueous solution to pH 1 to 2. The aqueous solution was extracted with ethyl acetate twice. The organic solution was concentrated and dried. The residue was dissolved in 3:1 CH3CN and water, and lyophilized to afford (R)-3-acetylamino-4-[3-(4′,5′-difluoro-2′-methoxy-biphenyl-4-yloxymethyl)-piperidin-1-yl]-4-oxo-butyric acid as white powder (50 mg, 65% yield). Mass spectrum [M+H]⁺: 491.

### Example 12

### (S)-3-tert-Butoxycarbonylamino-4-[3-(4′,5′-difluoro-2′-methoxy-biphenyl-4-yloxymethyl)-piperidin-1-yl]-4-oxo-butyric acid

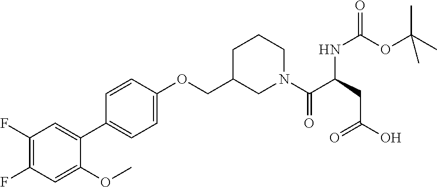

With a method similar as above, (S)-3-tert-butoxycarbonylamino-4-[3-(4′,5′-difluoro-2′-methoxy-biphenyl-4-yloxymethyl)-piperidin-1-yl]-4-oxo-butyric acid was prepared from 3-(4′,5′-difluoro-2′-methoxy-biphenyl-4-yloxymethyl)-piperidine and (S)-2-tert-butoxycarbonylamino-succinic acid 4-benzyl ester. Mass spectrum [M+H]⁺: 549.

### Example 13

### (S)-3-(Acetyl-methyl-amino)-4-[3-(4′,5′-difluoro-2′-methoxy-biphenyl-4-yloxymethyl)-piperidin-1-yl]-4-oxo-butyric acid

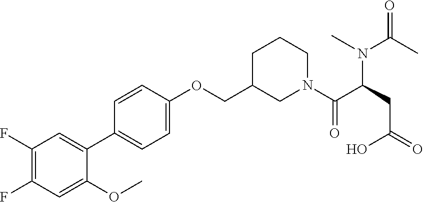

With a method similar as above, (S)-4-[3-(4′,5′-difluoro-2′-methoxy-biphenyl-4-yloxymethyl)-piperidin-1-yl]-3-[(9H-fluoren-9-ylmethoxycarbonyl)-methyl-amino]-4-oxo-butyric acid tert-butyl ester was prepared from 3-(4′,5′-difluoro-2′-methoxy-biphenyl-4-yloxymethyl)-piperidine and (S)-2-[(9H-fluoren-9-ylmethoxycarbonyl)-methyl-amino]-succinic acid 4-tert-butyl ester. (S)-4-[3-(4′,5′-difluoro-2′-methoxy-biphenyl-4-yloxymethyl)-piperidin-1-yl]-3-[(9H-fluoren-9-ylmethoxycarbonyl)-methyl-amino]-4-oxo-butyric acid tert-butyl ester (200 mg, 0.27 mmol) was then treated with 3 mL of 20% piperidine in dichloromethane at room temperature for 1 hour. The reaction was concentrated and dried under vacuum. The crude product was taken up in 3 mL of dichloromethane and treated with 0.2 mL of triethylamine and 0.2 mL of acetic anhydride. The reaction was stirred at room temperature for 1 h and then concentrated. The crude reaction mixture was purified by flash chromatography to afford (S)-3-(acetyl-methyl-amino)-4-[3-(4′,5′-difluoro-2′-methoxy-biphenyl-4-yloxymethyl)-piperidin-1-yl]-4-oxo-butyric acid tert-butyl ester as a colorless oil (90 mg, 60% yield).

(S)-3-(acetyl-methyl-amino)-4-[3-(4′,5′-difluoro-2′-methoxy-biphenyl-4-yloxymethyl)-piperidin-1-yl]-4-oxo-butyric acid tert-butyl ester (40 mg, 0.07 mmol) was stirred in 2 mL of formic acid at room temperature for 0.5 hour. The reaction mixture was concentrated, purified by preparative HPLC, and lyophilized to afford (S)-3-(acetyl-methyl-amino)-4-[3-(4′,5′-difluoro-2′-methoxy-biphenyl-4-yloxymethyl)-piperidin-1-yl]-4-oxo-butyric acid as an off-white powder (19 mg, 54% yield). Mass spectrum [M+H]⁺: 505.

### Example 14

4-[3-(4′,5′-Difluoro-2′-methoxy-biphenyl-4-yloxymethyl)-piperidine-1-carbonyl]-pyrrolidine-1,3-dicarboxylic acid 1-tert-butyl ester

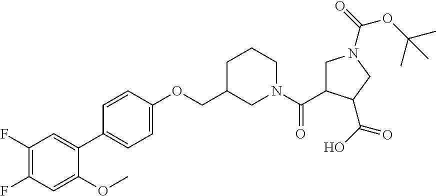

With a method similar as above, 4-[3-(4′,5′-difluoro-2′-methoxy-biphenyl-4-yloxymethyl)-piperidine-1-carbonyl]-pyrrolidine-1,3-dicarboxylic acid 1-tert-butyl ester was prepared from 3-(4′,5′-difluoro-2′-methoxy-biphenyl-4-yloxymethyl)-piperidine and 1-(tert-butoxycarbonyl)-4-(methoxycarbonyl)pyrrolidine-3-carboxylic acid. Mass spectrum [M+H]⁺: 575.

### Example 15

### 1-Acetyl-4-[3-(4′,5′-difluoro-2′-methoxy-biphenyl-4-yloxymethyl)-piperidine-1-carbonyl]-pyrrolidine-3-carboxylic acid

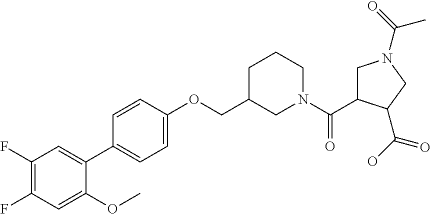

With a method similar as above, 1-acetyl-4-[3-(4′,5′-difluoro-2′-methoxy-biphenyl-4-yloxymethyl)-piperidine-1-carbonyl]-pyrrolidine-3-carboxylic acid was prepared from 4-[3-(4′,5′-difluoro-2′-methoxy-biphenyl-4-yloxymethyl)-piperidine-1-carbonyl]-pyrrolidine-1,3-dicarboxylic acid 1-tert-butyl ester and acetyl chloride. Mass spectrum [M+H]⁺: 517.

### Example 16

### 1-Carbamoyl-4-[3-(4′,5′-difluoro-2′-methoxy-biphenyl-4-yloxymethyl)-piperidine-1-carbonyl]-pyrrolidine-3-carboxylic acid

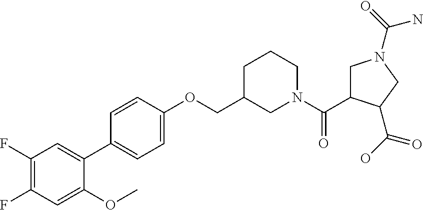

With a method similar as above, 1-carbamoyl-4-[3-(4′,5′-difluoro-2′-methoxy-biphenyl-4-yloxymethyl)-piperidine-1-carbonyl]-pyrrolidine-3-carboxylic acid was prepared from 3-(4′,5′-difluoro-2′-methoxy-biphenyl-4-yloxymethyl)-piperidine and trimethylsilyl isocyanate. Mass spectrum [M+H]⁺: 518.

### Example 17

### 3-[3-(4′,5′-Difluoro-2′-methoxy-biphenyl-4-yloxymethyl)-piperidin-1-yl]-2-methyl-3-oxo-propionic acid

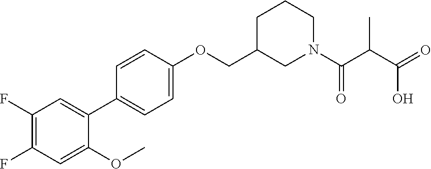

A mixture of 3-(4′,5′-difluoro-2′-methoxy-biphenyl-4-yloxymethyl)-piperidine (83 mg, 0.25 mmol), 2-methyl-malonic acid (89 mg, 0.75 mmol), 1-hydroxy-7azabenzotriazole (HOAT) (51 mg, 0.38 mmol) and 1-ethyl-2-(3-dimethylaminopropyl)carbodiimide hydrochloride (73 mg, 0.38 mmol) in anhydrous dichloromethane (5 mL) was stirred at room temperature overnight. The reaction mixture was mixed with water, acidified with 1 N HCl aqueous solution and the organic layer was separated. The organic solution was concentrated and purified by flash chromatography eluted with 0 to 40% methanol in dichloromethane. The product obtained was purified on preparative HPLC again to afford 3-[3-(4′,5′-difluoro-2′-methoxy-biphenyl-4-yloxymethyl)-piperidin-1-yl]-2-methyl-3-oxo-propionic acid as white powder (12 mg pure product isolated). Mass spectrum [M+H]⁺: 434.

### Example 18

2-[3-(4′,5′-Difluoro-2′-methoxy-biphenyl-4-yloxymethyl)-piperidine-1-carbonyl]-butyric acid

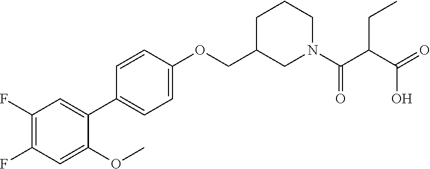

With a method similar as above, 2-[3-(4′,5′-difluoro-2′-methoxy-biphenyl-4-yloxymethyl)-piperidine-1-carbonyl]-butyric acid was prepared from 3-(4′,5′-difluoro-2′-methoxy-biphenyl-4-yloxymethyl)-piperidine and 2-ethyl-malonic acid. Mass spectrum [M+H]⁺: 448.

### Example 19

### 2-[3-(4′,5′-Difluoro-2′-methoxy-biphenyl-4-yloxymethyl)-piperidine-1-carbonyl]-3,3-dimethyl-butyric acid

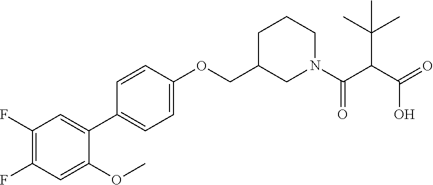

With a method similar as above, 2-[3-(4′,5′-difluoro-2′-methoxy-biphenyl-4-yloxymethyl)-piperidine-1-carbonyl]-3,3-dimethyl-butyric acid was prepared from 3-(4′,5′-difluoro-2′-methoxy-biphenyl-4-yloxymethyl)-piperidine and 2-tert-butyl-malonic acid. Mass spectrum [M+H]⁺: 476.

### Example 20

### 3-[3-(4′,5′-Difluoro-2′-methoxy-biphenyl-4-yloxymethyl)-piperidin-1-yl]-3-oxo-propionamide

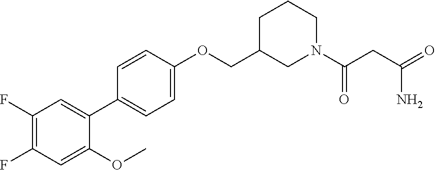

With a method similar as above, 3-[3-(4′,5′-difluoro-2′-methoxy-biphenyl-4-yloxymethyl)-piperidin-1-yl]-3-oxo-propionamide was prepared from 3-[3-(4′,5′-difluoro-2′-methoxy-biphenyl-4-yloxymethyl)-piperidin-1-yl]-3-oxo-propionic acid and ammonium chloride. Mass spectrum [M+H]⁺: 419.

### Example 21

### [3-(4′,5′-Difluoro-2′-methoxy-biphenyl-4-yloxymethyl)-piperidin-1-yl]-(1,1-dioxido-3-isothiazolidin-3-yl)-methanone

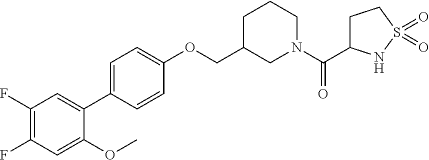

With a method similar as above, [3-(4′,5′-difluoro-2′-methoxy-biphenyl-4-yloxymethyl)-piperidin-1-yl]-(1,1-dioxido-3-isothiazolidin-3-yl)-methanone was prepared from 3-(4′,5′-difluoro-2′-methoxy-biphenyl-4-yloxymethyl)-piperidine and 1,1-dioxo-isothiazolidine-3-carboxylic acid (commercially available). Mass spectrum [M+H]⁺: 481.

### Example 22

### {Benzyl-[3-(4′,5′-difluoro-2′-methoxy-biphenyl-4-yloxymethyl)-piperidine-1-carbonyl]-amino}-acetic acid

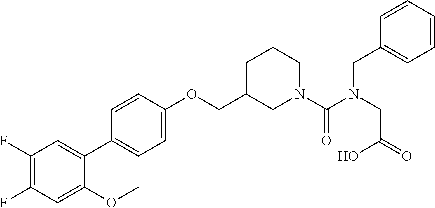

To a solution of 3-(4′,5′-difluoro-2′-methoxy-biphenyl-4-yloxymethyl)-piperidine (100 mg, 0.3 mmol) in 3 mL of anhydrous tetrahydrofuran at 0° C. was slowly added a 20% solution of phosgene in toluene (0.23 mL). The reaction mixture was warmed up to room temperature and stirred for 40 min and cooled down to 0° C. again. A solution of benzylamino-acetic acid ethyl ester (116 mg, 0.6 mmol) and triethylamine (0.3 mL) in 2 mL methylene chloride was added and stirred at 40° C. for 2 h and then at room temperature overnight. The solvent was evaporated. The residue was mixed with water and extracted with ethyl acetate. The organic solution was concentrated and purified by flash chromatography eluted with 0-60% ethyl acetate in hexane. The product obtained was treated with excess lithium hydroxide monohydrate (100 mg), and stirred in a mixed solvents of tetrahydrofuan:methanol:water (3:1:1) at 35° C. for 2 h. The reaction mixture was concentrated and then mixed with water, acidified with 1 N HCl aqueous solution to pH 1 to 2. The aqueous solution was extracted with ethyl acetate twice. The organic solution was concentrated and dried. The residue was dissolved in 3:1 CH3CN and water, and lyophilized to afford {benzyl-[3-(4′,5′-difluoro-2′-methoxy-biphenyl-4-yloxymethyl)-piperidine-1-carbonyl]-amino}-acetic acid as white powder (134 mg, 85% yield). Mass spectrum [M+H]⁺: 525.

### Example 23

### {Benzyl-[(R)-3-(4′,5′-difluoro-2′-methoxy-biphenyl-4-yloxymethyl)-piperidine-1-carbonyl]-amino}-acetic acid (or enantiomer)

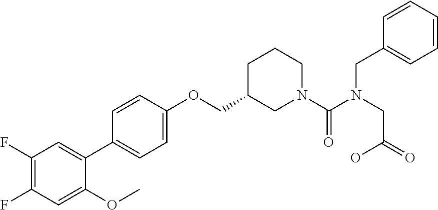

The racemic mixture {benzyl-[3-(4′,5′-difluoro-2′-methoxy-biphenyl-4-yloxymethyl)-piperidine-1-carbonyl]-amino}-acetic acid obtained above was separated by chiral SFC to afford {benzyl-[(R)-3-(4′,5′-difluoro-2′-methoxy-biphenyl-4-yloxymethyl)-piperidine-1-carbonyl]-amino}-acetic acid (or enantiomer). Mass spectrum [M+H]⁺: 525.

### Example 24

### {Benzyl-[(S)-3-(4′,5′-difluoro-2′-methoxy-biphenyl-4-yloxymethyl)-piperidine-1-carbonyl]-amino}-acetic acid (or enantiomer)

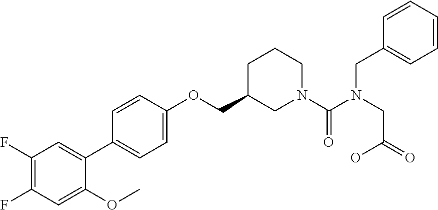

The racemic mixture {benzyl-[3-(4′,5′-difluoro-2′-methoxy-biphenyl-4-yloxymethyl)-piperidine-1-carbonyl]-amino}-acetic acid obtained above was separated by chiral SFC to afford {benzyl-[(S)-3-(4′,5′-difluoro-2′-methoxy-biphenyl-4-yloxymethyl)-piperidine-1-carbonyl]-amino}-acetic acid (or enantiomer). Mass spectrum [M+H]⁺: 525.

### Example 25

### {[3-(4′,5′-Difluoro-2′-methoxy-biphenyl-4-yloxymethyl)-piperidine-1-carbonyl]-methyl-amino}-acetic acid

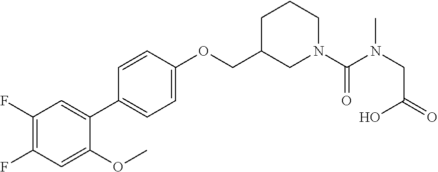

With a method similar as above {[3-(4′,5′-difluoro-2′-methoxy-biphenyl-4-yloxymethyl)-piperidine-1-carbonyl]-methyl-amino}-acetic acid was prepared from 3-(4′,5′-difluoro-2′-methoxy-biphenyl-4-yloxymethyl)-piperidine and methylamino-acetic acid ethyl ester. Mass spectrum [M+H]⁺: 449.

### Example 26

### {[3-(4′,5′-Difluoro-2′-methoxy-biphenyl-4-yloxymethyl)-pyrrolidine-1-carbonyl]-methyl-amino}-acetic acid

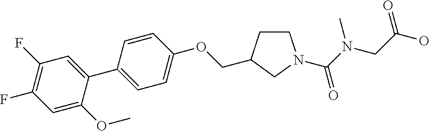

With a method similar as above, {[3-(4′,5′-difluoro-2′-methoxy-biphenyl-4-yloxymethyl)-pyrrolidine-1-carbonyl]-methyl-amino}-acetic acid was prepared from 3-(4′,5′-difluoro-2′-methoxy-biphenyl-4-yloxymethyl)-pyrrolidine and methylamino-acetic acid ethyl ester. Mass spectrum [M+H]⁺: 435.

### Example 27

### (S)-1-[3-(4′,5′-Difluoro-2′-methoxy-biphenyl-4-yloxymethyl)-piperidine-1-carbonyl]-pyrrolidine-2-carboxylic acid

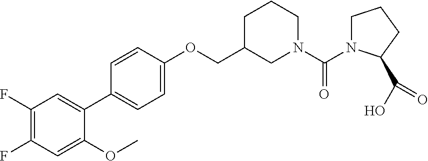

With a method similar as above, (S)-1-[3-(4′,5′-difluoro-2′-methoxy-biphenyl-4-yloxymethyl)-piperidine-1-carbonyl]-pyrrolidine-2-carboxylic acid was prepared from 3-(4′,5′-difluoro-2′-methoxy-biphenyl-4-yloxymethyl)-piperidine and L-proline methyl ester hydrochloride. Mass spectrum [M+H]⁺: 475.

### Example 28

### 3-{Benzyl-[3-(4′,5′-difluoro-2′-methoxy-biphenyl-4-yloxymethyl)-piperidine-1-carbonyl]-amino}-propionic acid

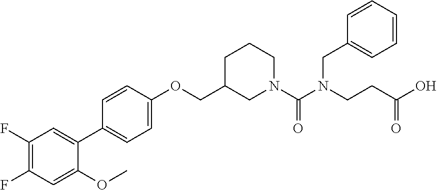

With a method similar as above, 3-{benzyl-[3-(4′,5′-difluoro-2′-methoxy-biphenyl-4-yloxymethyl)-piperidine-1-carbonyl]-amino}-propionic acid was prepared from 3-(4′,5′-difluoro-2′-methoxy-biphenyl-4-yloxymethyl)-piperidine and 3-benzylamino-propionic acid ethyl ester. Mass spectrum [M+H]⁺: 539.

### Example 29

### 3-{Benzyl-[3-(4′,5′-difluoro-2′-methoxy-biphenyl-4-yloxymethyl)-pyrrolidine-1-carbonyl]-amino}-propionic acid

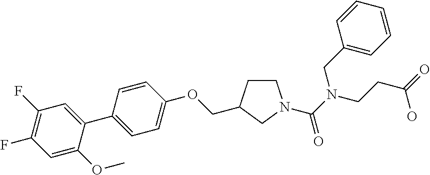

With a method similar as above, 3-{benzyl-[3-(4′,5′-difluoro-2′-methoxy-biphenyl-4-yloxymethyl)-pyrrolidine-1-carbonyl]-amino}-propionic acid was prepared from 3-(4′,5′-difluoro-2′-methoxy-biphenyl-4-yloxymethyl)-pyrrolidine and 3-benzylamino-propionic acid ethyl ester. Mass spectrum [M+H]⁺: 525.

### Example 30

### 3-(4′,5′-Difluoro-2′-methoxy-biphenyl-4-yloxymethyl)-piperidine-1-carboxylic acid methyl-(1H-tetrazol-5-ylmethyl)-amide

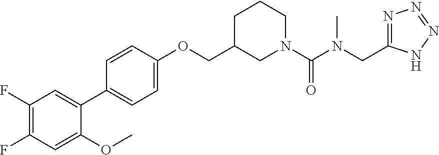

With a method similar as above, 3-(4′,5′-difluoro-2′-methoxy-biphenyl-4-yloxymethyl)-piperidine-1-carboxylic acid cyanomethyl-methyl-amide was prepared from 3-(4′,5′-difluoro-2′-methoxy-biphenyl-4-yloxymethyl)-piperidine and methylamino-acetonitrile. Mass spectrum [M+H]⁺: 430.

3-(4′,5′-Difluoro-2′-methoxy-biphenyl-4-yloxymethyl)-piperidine-1-carboxylic acid cyanomethyl-methyl-amide (174 mg, 0.41 mmol) was then dissolved in 6 mL of toluene and treated with sodium azide (81 mg, 1.24 mmol, 3 eq) and triethylamine hydrochloride (171 mg, 1.24 mmol, 3 eq). The reaction was stirred at 100° C. for overnight. The solvent was removed, and the residue was mixed with water and extracted with EtOAc (2×). The organic layer was washed with saturated NaHCO₃, and then discarded. The NaHCO₃ washing solution was combined with the aqueous layer and acidified with 1 N HCl to pH 1 to 2. The solution was then extracted with EtOAc (2×). The organic solution was concentrated. The residue was dissolved in 3:1 CH3CN: water, and lyophilized. 3-(4′,5′-difluoro-2′-methoxy-biphenyl-4-yloxymethyl)-piperidine-1-carboxylic acid methyl-(1H-tetrazol-5-ylmethyl)-amide was obtained as light yellow powder (115 mg, 60% yield). Mass spectrum [M+H]⁺: 473.

### Example 31

### 3-(4′,5′-Difluoro-2′-methoxy-biphenyl-4-yloxymethyl)-piperidine-1-carboxylic acid (2-methanesulfonylamino-2-oxo-ethyl)-methyl-amide

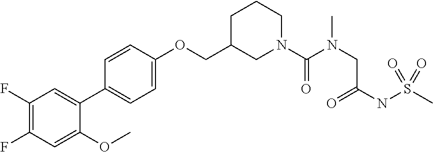

{[3-(4′,5′-Difluoro-2′-methoxy-biphenyl-4-yloxymethyl)-piperidine-1-carbonyl]-methyl-amino}-acetic acid prepared as above (152 mg, 0.34 mmol) was dissolved in dichloromethane (5 mL) and cooled down to 0° C. and was treated with N,N′-carbonyldiimidazole (72 mg, 0.44 mmol). After stirred at 0° C. for 2 h, 1,8-diazabicyclo[5.4.0]undec-7-ene (76 uL, 0.51 mmol) and methanesulfonamide (48 mg, 0.51 mmol) were added to the reaction. The mixture was stirred at 23° C. for 2 h before 0.03 mL of acetic acid was added. The reaction was continually stirred at room temperature for another half hour and then was concentrated. The mixture was purified by flash chromatography eluted with 90/9/1 dichloromethane/methanol/acetic acid. After lyophilization, 3-(4′,5′-difluoro-2′-methoxy-biphenyl-4-yloxymethyl)-piperidine-1-carboxylic acid (2-methanesulfonyl amino-2-oxo-ethyl)-methyl-amide was obtained as off-white powder (147 mg, 83% yield). Mass spectrum [M+H]⁺: 526.

### Example 32

### (S)-3-(4′,5′-Difluoro-2′-methoxy-biphenyl-4-yloxymethyl)-piperidine-1-carboxylic acid (2-methanesulfonylamino-2-oxo-ethyl)-methyl-amide

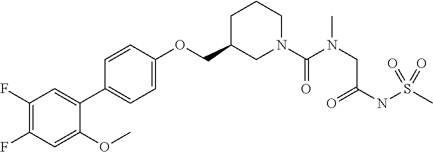

The racemic mixture 3-(4′,5′-difluoro-2′-methoxy-biphenyl-4-yloxymethyl)-piperidine-1-carboxylic acid (2-methanesulfonyl amino-2-oxo-ethyl)-methyl-amide obtained above was separated by chiral SFC to afford (S)-3-(4′,5′-difluoro-2′-methoxy-biphenyl-4-yloxymethyl)-piperidine-1-carboxylic acid (2-methanesulfonylamino-2-oxo-ethyl)-methyl-amide (or enantiomer). [α]_{d}²⁵=+17.6 in DMSO, 5 mg/mL.

### Example 33

### (R)-3-(4′,5′-Difluoro-2′-methoxy-biphenyl-4-yloxymethyl)-piperidine-1-carboxylic acid (2-methanesulfonylamino-2-oxo-ethyl)-methyl-amide

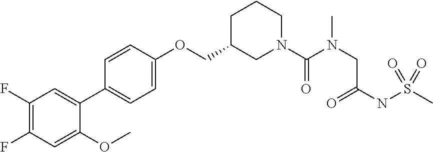

The racemic mixture 3-(4′,5′-difluoro-2′-methoxy-biphenyl-4-yloxymethyl)-piperidine-1-carboxylic acid (2-methanesulfonyl amino-2-oxo-ethyl)-methyl-amide obtained above was separated by chiral SFC to afford (R)-3-(4′,5′-difluoro-2′-methoxy-biphenyl-4-yloxymethyl)-piperidine-1-carboxylic acid (2-methanesulfonylamino-2-oxo-ethyl)-methyl-amide (or enantiomer). [α]_{d}²⁵=−2.8 in DMSO, 5 mg/mL.

### Example 34

### 3-(4′,5′-Difluoro-2′-methoxy-biphenyl-4-yloxymethyl)-piperidine-1-carboxylic acid benzyl-(2-methanesulfonylamino-2-oxo-ethyl)-amide

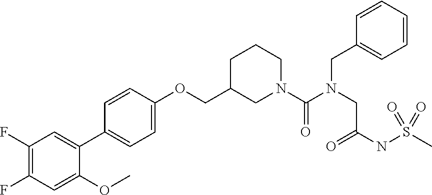

With a method similar as above, 3-(4′,5′-difluoro-2′-methoxy-biphenyl-4-yloxymethyl)-piperidine-1-carboxylic acid benzyl-(2-methanesulfonylamino-2-oxo-ethyl)-amide was prepared from {benzyl-[3-(4′,5′-difluoro-2′-methoxy-biphenyl-4-yloxymethyl)-piperidine-1-carbonyl]-amino}-acetic acid and methanesulfonamide. Mass spectrum [M+H]⁺: 602.

### Example 35

### 3-(4′,5′-Difluoro-2′-methoxy-biphenyl-4-yloxymethyl)-pyrrolidine-1-carboxylic acid benzyl-(2-methanesulfonylamino-2-oxo-ethyl)-amide

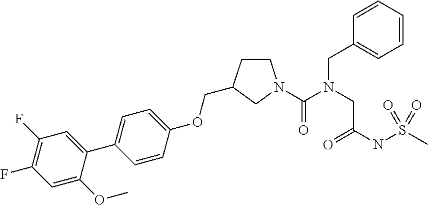

With a method similar as above, 3-(4′,5′-difluoro-2′-methoxy-biphenyl-4-yloxymethyl)-pyrrolidine-1-carboxylic acid benzyl-(2-methanesulfonylamino-2-oxo-ethyl)-amide was prepared from 3-(4′,5′-difluoro-2′-methoxy-biphenyl-4-yloxymethyl)-pyrrolidine, benzylamino-acetic acid and methanesulfonamide. Mass spectrum [M+H]⁺: 588.

### Example 36

### 3-(4′,5′-Difluoro-2′-methoxy-biphenyl-4-yloxymethyl)-pyrrolidine-1-carboxylic acid (2-methanesulfonylamino-2-oxo-ethyl)-methyl-amide

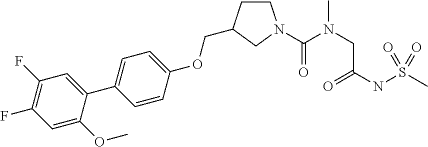

With a method similar as above, 3-(4′,5′-difluoro-2′-methoxy-biphenyl-4-yloxymethyl)-pyrrolidine-1-carboxylic acid (2-methanesulfonylamino-2-oxo-ethyl)-methyl-amide was prepared from {[3-(4′,5′-difluoro-2′-methoxy-biphenyl-4-yloxymethyl)-pyrrolidine-1-carbonyl]-methyl-amino}-acetic acid and methanesulfonamide. Mass spectrum [M+H]⁺: 512.

### Example 37

### Glycogen Synthase (GS) Assay

The following tests were carried out in order to determine the activity of the compounds of formula (I).

Twelve μL per well of substrate solution containing glycogen (4.32 mg/mL), 2.67 mM UDP-glucose, 21.6 mM phospho(enol)pyruvate and 2.7 mM NADH in 30 mM glycylglycine, pH 7.3 buffer was added into a polystyrene 384-well assay plate (BD Biosciences).

Compound solutions (8 μL/well) at various concentrations (0-300 μM) were added to the assay plate (columns 5-24). Compound solution contains 30 mM glycylglycine, pH 7.3, 40 mM KCl, 20 mM MgC1₂, 9.2% DMSO, with (columns 15-24) or without (columns 5-14) 20 mM glucose 6-phosphate.

Enzyme solution (12 μL/well) containing glycogen synthase (16.88 μg/mL), pyruvate kinase (0.27 mg/mL), lactate dehydrogenase (0.27 mg/mL) in 50 mM Tris-HCl, pH 8.0, 27 mM DTT and bovine serum albumin (BSA, 0.2 mg/mL) was added to the assay plate (columns 3-24). As a blank control, enzyme solution without glycogen synthase was added into the top half wells of columns 1-2. To the bottom half wells of columns 1-2 were added a known activator, glucose 6-phosphate (at final concentration 5 mM) in addition to the enzyme solution. The reaction mixture was incubated at room temperature. The assay plate was then read for absorbance at 340 nm on an Envision reader every 3 minutes up to a total of 15 minutes.

The enzyme activity (with or without compound) was calculated by the reaction rate and represented by the optical density change (SOD) per minute. Percent stimulation of glycogen synthase activity by a compound at various concentrations was calculated by the following formula:

% stimulation=100*Rs/Rt,

where Rs is the reaction rate of the enzyme in the presence of compound and Rt is the reaction rate of the enzyme in the absence of compound.

SC₂₀₀ is defined as the compound concentration that is needed to stimulate 200% of the enzyme activity. EC₅₀ is defined as the compound concentration that is needed to give 50% maximum activation.

Compounds from Example 1 through Example 36 were assayed according to assay procedures described above and the results are listed in Table 1 below:

TABLE 1
Glycogen Synthase Activation Potency

| Example Number | GS SC₂₀₀ (μM) | GS EC₅₀ (μM) |
|---|---|---|
| 1 | 1.1 | 3.4 |
| 2 | 0.05 | 0.51 |
| 3 | 0.21 | 0.35 |
| 4 | 1.18 | 1.74 |
| 5 | 0.99 | 6.42 |
| 6 | 2.37 | 5.54 |
| 7 | 1.55 | 6.28 |
| 8 | 2.35 | 8.03 |
| 9 | 2.22 | 6.26 |
| 10 | 1.51 | 5.71 |
| 11 | 0.42 | 1.93 |
| 12 | 3.74 | 11.07 |
| 13 | 2.12 | 12.29 |
| 14 | 1.94 | 3.72 |
| 15 | 3.65 | 5.78 |
| 16 | 0.42 | 6.48 |
| 17 | 0.29 | 0.68 |
| 18 | ND | 0.98 |
| 19 | 2.21 | 5.35 |
| 20 | 6.34 | 12.74 |
| 21 | ND | 7.3 |
| 22 | 0.12 | 2.65 |
| 23 | 0.23 | 0.95 |
| 24 | 1.99 | 4.76 |
| 25 | 0.02 | 0.29 |
| 26 | 5.97 | 24.74 |
| 27 | 0.22 | 1.29 |
| 28 | 2.52 | 6.29 |
| 29 | 2.55 | 10.38 |
| 30 | 1.38 | 8.29 |
| 31 | 0.11 | 0.38 |
| 32 | 0.06 | 0.2 |
| 33 | 0.41 | 2.03 |
| 34 | 0.07 | 0.52 |
| 35 | 0.28 | 0.82 |
| 36 | 0.8 | 7.68 |

ND = not determined.

It is to be understood that the invention is not limited to the particular embodiments of the invention described above, as variations of the particular embodiments may be made and still fall within the scope of the appended claims.

What is claimed is:

1. A compound of Formula (I): 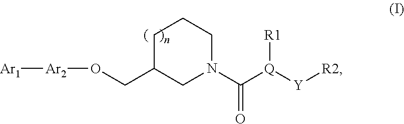
wherein:
Ar₁ is phenyl, mono-, bi- or tri-substituted independently with halogen, lower alkyl or alkoxy;
Ar₂ is phenyl unsubstituted or substituted with halogen;
Q is CH, N, unsubstituted cycloalkyl, unsubstituted heterocycloalkyl or dioxidoisothiazolidine;
Y is CH₂, carbonyl or absent;
R1 is H, lower alkyl, unsubstituted or mono-, bi- or tri-substituted with halogen, —NR3R4, —C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1,
or a pharmaceutically acceptable salt thereof.

2. The compound according to claim 1, wherein:
Ar₁ is phenyl, mono-, bi- or tri-substituted independently with halogen, lower alkyl or alkoxy;
Ar₂ is phenyl unsubstituted or substituted with halogen;
Q is CH;
Y is CH₂, carbonyl or absent;
R1 is H, unsubstituted lower alkyl, —NR3R4, —C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1.

3. The compound according to claim 1, wherein:
Ar₁ is phenyl, mono-, bi- or tri-substituted independently with halogen, lower alkyl or alkoxy;
Ar₂ is phenyl unsubstituted or substituted with halogen;
Q is N;
Y is CH₂, carbonyl or absent;
R1 is H, unsubstituted lower alkyl, —NR3R4, —C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1.

4. The compound according to claim 1, wherein:
Ar₁ is phenyl, mono-, bi- or tri-substituted independently with halogen, lower alkyl or alkoxy;
Ar₂ is phenyl unsubstituted or substituted with halogen;
Q is unsubstituted cycloalkyl;
Y is CH₂, carbonyl or absent;
R1 is H, unsubstituted lower alkyl, —NR3R4, —C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1.

5. The compound according to claim 1, wherein:
Ar₁ is phenyl, mono-, bi- or tri-substituted independently with halogen, lower alkyl or alkoxy;
Ar₂ is phenyl unsubstituted or substituted with halogen;
Q is unsubstituted heterocycloalkyl;
Y is CH₂, carbonyl or absent;
R1 is H, unsubstituted lower alkyl, —NR3R4, —C(O)OC(CH₃)₃, —C(O)CH₃, —C(O)NH₂, —CH₂-phenyl or absent;
R2 is H, —NOCH₃, —NOH, —C(O)NH₂, —(CH₂)ₘCOOH, —C(O)NSO₂CH₃ or 1H-tetrazole;
R3 is H or lower alkyl;
R4 is —C(O)CH₃ or —C(O)OC(CH₃)₃;
n is 0 or 1; and
m is 0 or 1.

6. The compound according to claim 1, wherein Ar₁ is difluoromethoxy phenyl.

7. The compound according to claim 1, wherein Ar₂ is unsubstituted phenyl.

8. The compound according to claim 1, wherein Q is CH or N.

9. The compound according to claim 1, wherein Q is N.

10. The compound according to claim 1, wherein Q is piperidine.

11. The compound according to claim 1, wherein Y is CH₂.

12. The compound according to claim 1, wherein Y is carbonyl or absent.

13. The compound according to claim 1, wherein R1 is H, lower alkyl, tert-butoxycarbonylamino, acetylamino, acetyl-methyl-amino, —C(O)OC(CH₃)₃, —C(O)CH₃, —C(O)NH₂, —CH₂-phenyl or absent.

14. The compound according to claim 1, wherein R1 is H.

15. The compound according to claim 1, wherein R1 is methyl, ethyl or tert-butyl.

16. The compound according to claim 1, wherein R1 is tert-butoxycarbonylamino, acetylamino or acetyl-methyl-amino.

17. The compound according to claim 1, wherein R1 is —C(O)OC(CH₃)₃, —C(O)CH₃, —C(O)NH₂ or —CH₂-phenyl.

18. The compound according to claim 1, wherein R1 is absent.

19. The compound according to claim 1, wherein R2 is —NOCH₃, —NOH, —C(O)NH₂, —(CH₂)ₘCOOH, —C(O)NSO₂CH₃ or 1H-tetrazole.

20. The compound according to claim 1, wherein R3 is H, methyl or ethyl.

21. The compound according to claim 1, wherein R4 is —C(O)CH₃.

22. The compound according to claim 1, wherein R4 is —C(O)OC(CH₃)₃.

23. The compound according to claim 1, wherein n is 0.

24. The compound according to claim 1, wherein n is 1.

25. The compound according to claim 1, wherein m is 0.

26. The compound according to claim 1, wherein m is 1.

27. The compound according to claim 1, wherein said compound is:
1-[3-(4′,5′-Difluoro-2′-methoxy-biphenyl-4-yloxymethyl)-piperidine-1-carbonyl]-cyclopropanecarboxylic acid;
3-[3-(4′,5′-Difluoro-2′-methoxy-biphenyl-4-yloxymethyl)-piperidin-1-yl]-3-oxo-propionic acid;
3-[(S)-3-(4′,5′-Difluoro-2′-methoxy-biphenyl-4-yloxymethyl)-piperidin-1-yl]-3-oxo-propionic acid;
3-[(R)-3-(4′,5′-Difluoro-2′-methoxy-biphenyl-4-yloxymethyl)-piperidin-1-yl]-3-oxo-propionic acid;
3-[3-(4′,5′-Difluoro-2′-methoxy-biphenyl-4-yloxymethyl)-piperidin-1-yl]-N-methoxy-3-oxo-propionamide;
3-[3-(4′,5′-Difluoro-2′-methoxy-biphenyl-4-yloxymethyl)-piperidin-1-yl]-N-hydroxy-3-oxo-propionamide;
2-[3-(4′,5′-Difluoro-2′-methoxy-biphenyl-4-yloxymethyl)-piperidine-1-carbonyl]-cyclopentanecarboxylic acid;
(R)(+)-4-[3-(4′,5′-Difluoro-2′-methoxy-biphenyl-4-yloxymethyl)-piperidin-1-yl]-3-methyl-4-oxo-butyric acid;
3-(4′,5′-Difluoro-2′-methoxy-biphenyl-4-yloxymethyl)-piperidine-1-carboxylic acid amide;
(R)-3-tert-Butoxycarbonylamino-4-[3-(4′,5′-difluoro-2′-methoxy-biphenyl-4-yloxymethyl)-piperidin-1-yl]-4-oxo-butyric acid;
(R)-3-Acetylamino-4-[3-(4′,5′-difluoro-2′-methoxy-biphenyl-4-yloxymethyl)-piperidin-1-yl]-4-oxo-butyric acid;
(S)-3-tert-Butoxycarbonylamino-4-[3-(4′,5′-difluoro-2′-methoxy-biphenyl-4-yloxymethyl)-piperidin-1-yl]-4-oxo-butyric acid;
(S)-3-(Acetyl-methyl-amino)-4-[3-(4′,5′-difluoro-2′-methoxy-biphenyl-4-yloxymethyl)-piperidin-1-yl]-4-oxo-butyric acid;
4-[3-(4′,5′-Difluoro-2′-methoxy-biphenyl-4-yloxymethyl)-piperidine-1-carbonyl]-pyrrolidine-1,3-dicarboxylic acid 1-tert-butyl ester;
1-Acetyl-4-[3-(4′,5′-difluoro-2′-methoxy-biphenyl-4-yloxymethyl)-piperidine-1-carbonyl]-pyrrolidine-3-carboxylic acid;
1-Carbamoyl-4-[3-(4′,5′-difluoro-2′-methoxy-biphenyl-4-yloxymethyl)-piperidine-1-carbonyl]-pyrrolidine-3-carboxylic acid;
3-[3-(4′,5′-Difluoro-2′-methoxy-biphenyl-4-yloxymethyl)-piperidin-1-yl]-2-methyl-3-oxo-propionic acid;
2-[3-(4′,5′-Difluoro-2′-methoxy-biphenyl-4-yloxymethyl)-piperidine-1-carbonyl]-butyric acid;
2-[3-(4′,5′-Difluoro-2′-methoxy-biphenyl-4-yloxymethyl)-piperidine-1-carbonyl]-3,3-dimethyl-butyric acid; or
3-[3-(4′,5′-Difluoro-2′-methoxy-biphenyl-4-yloxymethyl)-piperidin-1-yl]-3-oxo-propionamide.

28. The compound according to claim 1, wherein said compound is:
[3-(4′,5′-Difluoro-2′-methoxy-biphenyl-4-yloxymethyl)-piperidin-1-yl]-(1,1-dioxido-3-isothiazolidin-3-yl)-methanone;
{Benzyl-[3-(4′,5′-difluoro-2′-methoxy-biphenyl-4-yloxymethyl)-piperidine-1-carbonyl]-amino}-acetic acid;
{Benzyl-[(R)-3-(4′,5′-difluoro-2′-methoxy-biphenyl-4-yloxymethyl)-piperidine-1-carbonyl]-amino}-acetic acid;
{Benzyl-[(S)-3-(4′,5′-difluoro-2′-methoxy-biphenyl-4-yloxymethyl)-piperidine-1-carbonyl]-amino}-acetic acid;
{[3-(4′,5′-Difluoro-2′-methoxy-biphenyl-4-yloxymethyl)-piperidine-1-carbonyl]-methyl-amino}-acetic acid;
{[3-(4′,5′-Difluoro-2′-methoxy-biphenyl-4-yloxymethyl)-pyrrolidine-1-carbonyl]-methyl-amino}-acetic acid;
(S)-1-[3-(4′,5′-Difluoro-2′-methoxy-biphenyl-4-yloxymethyl)-piperidine-1-carbonyl]-pyrrolidine-2-carboxylic acid;
3-{Benzyl-[3-(4′,5′-difluoro-2′-methoxy-biphenyl-4-yloxymethyl)-piperidine-1-carbonyl]-amino}-propionic acid;
3-{Benzyl-[3-(4′,5′-difluoro-2′-methoxy-biphenyl-4-yloxymethyl)-pyrrolidine-1-carbonyl]-amino}-propionic acid;
3-(4′,5′-Difluoro-2′-methoxy-biphenyl-4-yloxymethyl)-piperidine-1-carboxylic acid methyl-(1H-tetrazol-5-ylmethyl)-amide;
3-(4′,5′-Difluoro-2′-methoxy-biphenyl-4-yloxymethyl)-piperidine-1-carboxylic acid (2-methanesulfonylamino-2-oxo-ethyl)-methyl-amide;
(S)-3-(4′,5′-Difluoro-2′-methoxy-biphenyl-4-yloxymethyl)-piperidine-1-carboxylic acid (2-methanesulfonylamino-2-oxo-ethyl)-methyl-amide;
(R)-3-(4′,5′-Difluoro-2′-methoxy-biphenyl-4-yloxymethyl)-piperidine-1-carboxylic acid (2-methanesulfonylamino-2-oxo-ethyl)-methyl-amide;
3-(4′,5′-Difluoro-2′-methoxy-biphenyl-4-yloxymethyl)-piperidine-1-carboxylic acid benzyl-(2-methanesulfonylamino-2-oxo-ethyl)-amide;
3-(4′,5′-Difluoro-2′-methoxy-biphenyl-4-yloxymethyl)-pyrrolidine-1-carboxylic acid benzyl-(2-methanesulfonylamino-2-oxo-ethyl)-amide; or
3-(4′,5′-Difluoro-2′-methoxy-biphenyl-4-yloxymethyl)-pyrrolidine-1-carboxylic acid (2-methanesulfonylamino-2-oxo-ethyl)-methyl-amide.

29. A pharmaceutical composition, comprising a therapeutically effective amount of a compound according to claim 1 and a pharmaceutically acceptable carrier and/or adjuvant.
